(19) United States
(12) Patent Application Publication
(10) Pub. No.: US 2010/0152154 A1
(43) Pub. Date: Jun. 17, 2010

(54) NOVEL PLATINUM COMPOUNDS FOR TREATING CANCER

(76) Inventors: Jiajiu Shaw, Ann Arbor, MI (US); Fenlai Tan, Troy, MI (US)

Correspondence Address: JiaJiu Shaw, 3166 Shamrock Ct, Ann Arbor, MI, 48105, US

(21) Appl. No.: 12/636,713

(22) Filed: Dec. 12, 2009

Related U.S. Application Data
(60) Provisional application No. 61/122,384, filed on Dec. 14, 2008.

(51) Int. Cl. A61K 31/555; C07D 231/12; A61P 35/00; A61P 31/18

(52) U.S. Cl. 514/184; 548/109

(57) ABSTRACT

A series of platinum compounds and methods of treating cancer by said platinum compounds are disclosed.

### FIELD OF INVENTION

This invention relates to a series of novel platinum compounds and to methods of treating cancer by said platinum compounds.

### BACKGROUND

Cisplatin, cis-diamminedichloroplatinum(II) (commercial product name is Platinol®), has been used as a chemotherapeutic agent for more than twenty years since the discovery of its anti-tumor activity by B. Rosenberg (U.S. Pat. No. 4,177,263). The Oct. 23, 1995 issue of Chemical & Engineering News reported, “Cisplatin was first synthesized in the 1800s, but its anticancer activity was not discovered until the 1960s. In 1979, it was approved by the Food and Drug Administration (FDA) for clinical treatment of testicular and ovarian tumors and cancers of the head and neck”. The Physician's Desk Reference states that cisplatin can be used to treat testicular cancer, ovarian cancer, and bladder cancer.

As a first generation platinum drug, cisplatin is still being widely used because of its efficacy. However, it is far from being a perfect anticancer drug. Carboplatin (Paraplatin®) was approved by the FDA as the second platinum drug; it appears to have a better therapeutic index than cisplatin and is more widely prescribed than cisplatin. However, carboplatin still has significant toxicity and can incur drug resistance from repeat treatment. The third platinum drug, oxaliplatin, has been on European market for several years and was also approved in the US in recent years. Although it appears to have lower toxicity than cisplatin and carboplatin, its efficacy is also lower and oxaliplatin is typically administered in combination with leucovorin (folinic acid) and fluorouracil (also known as FOLFOX: FOL: folinic acid; F: fluorouracil; OX: oxaliplatin) for the treatment of colorectal cancer. Therefore, there is a continued interest in finding significantly improved platinum drugs.

Structures of cisplatin, carboplatin, and oxaliplatin are shown below:

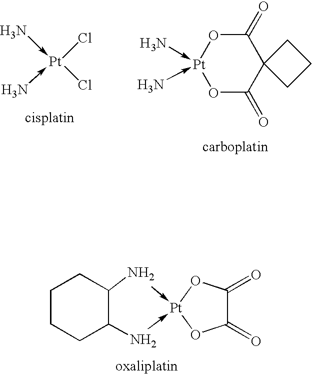

Cisplatin is known to function as an inhibitor to the DNA replication process; with the hampered ability to replicate, cancer cells eventually die. It is believed that the inhibition is due to the intra-strand cross-linkage between cisplatin and DNA through the two labile Pt—Cl bonds, especially during the DNA replication process. It is also known that when the two Pt—Cl bonds in cisplatin are switched to the trans position (i.e., trans-diamminedichloroplatinum(II)), the anticancer effect essentially disappears.

It is therefore logical for scientists to focus on modifications of the cis-form. Therefore, most platinum compounds have been made basically by modifying the two electron-donating NH₃ groups in cis-position. This type of modification is not very innovative in terms of changing the way platinum compound inhibits DNA replication.

One innovative way to improve platinum compounds is to use a dynamic mechanism to circumvent the efficacy/toxicity issue. Examples of this type of compounds are represented by several patents by J. Shaw (U.S. Pat. Nos. 6,534,096, 6,548,541, and 7,160,908).

Another less obvious but interesting way to improve platinum compounds is to use a 3-N coordination approach. Examples are shown by Hollis et al. (J. Med. Chem. 1989, 32, 128-136). As shown by Shaw (U.S. Pat. No. 5,922,689), the 3-N platinum compound made between cisplatin and guanosine (my patent) is effective in inhibiting the cell growth of MCF-7 and MDA-MB-231 and its toxicity to normal human mammary cells is lower than cisplatin in vitro. Although there is no definite evidence as to how a 3-N platinum compound works in inhibiting the replication of DNA, it is believed that the 3^{rd} nitrogen group may be wedged into the DNA being replicated and stays there long enough to substantiate the effect of inhibition on DNA replication. Therefore, using a building block of DNA, such as guanosine, to be the 3^{rd} nitrogen group would work well for the anticancer activity of such a platinum compound.

### DETAILED DESCRIPTION OF THE PRESENT INVENTION

Disclosed in the present invention is a series of novel platinum compounds which can be represented by the following general formula:

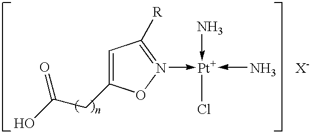

wherein n=0 or 1, R is H, CH₃, or CH₂CH₃, and X is Cl, Br, or NO₃.
These platinum compounds can be made as adducts between cisplatin and the following ligands individually at a 1:1 mole ratio.

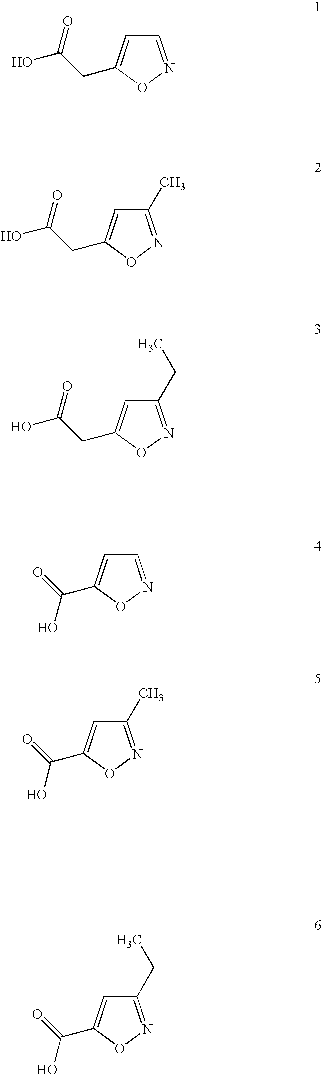

Although these type of compound (3-N platinum compounds) are known, these adducts disclosed herein are new to the best of our knowledge. Therefore, they are novel. In addition, these platinum compounds comprise a 3^{rd} nitrogen group which is not a building block of DNA. Therefore, these compounds are not obvious and not anticipated to have significant anticancer activity similar to that of cisplatin.

Based on platinum chemistry, it is clear that the nitrogen on isoxazole ring provides a lone pair electron and forms a coordination bond with Pt⁺² ion to form the 3-N platinum compound. Such a coordination bond has been published by J. Davis (J. Chem. Soc., Perkins Trans. 1, 1998, 3139-3140) and E. Horn et al. (Z. Kristallogr. NCS 220, 2005, 1-2).

These 3-N platinum compounds can be synthesized by well established method by Hollis et al. (J. Med. Chem. 1989, 32, 128-136) or by Shaw (U.S. Pat. No. 5,922,689). The method comprises the following general steps: (1) dissolving equal moles of cis-diamminedichloroplatinum(II) in a suitable amount of DI water to make a cisplatin solution, (2) adding an equal moles of one of the ligands in the cisplatin solution prepared in step 1 and mix for a suitable time, (3) removing any precipitate, and (4) concentrating and crystallizing the platinum compound in diluted HCl or methanol. The preferred suitable temperature is between 15° C. and 95° C. and the more preferred temperature is between 30° C. and 60° C. It is preferable to conduct the reaction under yellow light. It is understandable that reasonable and common-sense modifications may be necessary to increase the purity of the product and/or efficiency of the process.

A specific example for making one of the platinum compounds by this method is shown below:

### Example 1

### Synthesis of JP0916

1. Weigh cisplatin (mw 300) (2 m mole or 600 mg) and place in 150 ml DI water. Mix continuously at ca. 60° C. till cisplatin is all dissolved.
2. Weigh ligand 2 (3-methyl-5-isoxazoleacetic acid, mw 141.12) (2 m mole or 282 mg) and gradually add it the cisplatin solution until it is all dissolved. Continue heating and mixing for 2 hr. Adding 0.1 N NaOH to raise the pH to about 6 can increase the yield.
3. Cool the aliquot and filter off the precipitate.
4. Reduce the filtrate volume under vacuum to ca. 10-20 ml and a lot of nice yellow needle-like crystals appear.
5. Filter and keep the crystals, and let the crystals dry in air.
6. Use diluted HCl to recrystallize and obtain the pure product (herein after compound JP0916).
Note: All containers must be clean and rinsed with deionized water before use. All steps should be conducted under yellow light as much as possible.

Another method for synthesizing these 3-N platinum compounds is by the AgNO₃/DMF method as used by Lippert et al. (Inorg. Chim. Acta 1979, 37(1), L495) and by Hollis et al. (J. Med. Chem. 1989, 32, 128-136). Reasonable modifications of this method (e.g., change the solvent from DMF to water) can also be made to make these platinum compounds.

In addition to the novel molecular structures, all of these platinum compounds have another unique characteristic. Because of the carboxyl group on the ligand, these compounds tend to have better solubility in normal biological pH environment (pH˜7.4) as compared to in lower pH environment generally associated with solid tumor. This characteristic makes it easier for these platinum compounds to be excreted from the normal tissues, thus lowers the potential side effects.

Surprisingly, the platinum compound, JP0916, showed very impressive anticancer effect in vitro, significantly better than cisplatin for most of the cancer cell lines studied. The results are shown in Example 2 below:

### Example 2

### To Determine the In Vitro IC₅₀ Values on Ten Human Cancer Cell Lines

### Cell Lines and Media

HepG2 and PLC/PRF/5 cells are cultured in DMEM medium. Hep3B cells are cultured in MEM medium. NCI-H460 and 786-0 cells are cultured in RPMI 1640 medium. Caki-1, HCT-116, and SK-OV-3 cells are cultured in McCoy's 5A medium. MDA-MB-231 cells are cultured in L15 medium. All cells are cultured in corresponding media supplemented with 10% FBS, at 37° C., 5% CO₂, and 95% humidity. All media are purchased from GIBCO.

### Reagents

CellTiter 96® Aqueous MTS reagent powder (Cat. No. G1112, Promega)
Phenazine methosulfate (PMS) (Cat. No. P9625, Sigma)

### Test Articles

JP0916 and cisplatin

### Equipment

SpectraMAX plus microplate spectrophotometer (Molecular Devices Corp), incubator, reverse microscope

### Determination of IC₅₀ Values

Cells are harvested during the logarithmic growth period and counted with hemocytometer. Add 90 μl cell suspensions to 96-well plates (triplicates for each cell concentration), the final cell densities are 2×10⁴, 1×10⁴, or 5×10³ cells/well. Prepare a series of drug concentrations for each of JP0916 and cisplatin and dispense 10 μl drug solution in each well (triplicate for each drug concentration). Incubate the plate for 48 hr then measure by MTS assay (490 nm).

### Results:

IC₅₀ values (μM)

| | Liver Cancer | | | Kidney Cancer | |
|---|---|---|---|---|---|
| Cell Line | HepG2 | Hep3B | PC/PRF/5 | 786-0 | Caki-1 |
| JP0916 | 11.69 | 1.165 | 11.02 | 2.174 | 5.690 |
| Cisplatin | 25.63 | 3.075 | 21.77 | 3.541 | 10.47 |

| | Breast Cancer | Colon Cancer | NSCLC | Ovarian Cancer |
|---|---|---|---|---|
| Cell Line | MDA-MB-231 | HCT-116 | NCI-H460 | SK-OV-3 |
| JP0916 | 106.3 | 2.965 | 4.566 | 9.297 |
| cisplatin | 53.18 | 10.62 | 8.562 | 21.62 |

As shown in the above tables, JP0916 is surprisingly more potent than cisplatin in vitro on all cell lines except MDA-MB-231 under current experimental condition.

Therefore, the present invention discloses a method for treating cancer comprising administering to a cancer patient a therapeutically effective amount of one of the above platinum compounds in pharmaceutically acceptable dosage forms. Said cancer comprises colon cancer, ovarian cancer, cervical cancer, breast cancer, lung cancer, head and neck cancer, brain cancer, liver cancer, leukemia, pancreatic cancer, skin cancer, prostate cancer, and stomach cancer. The dosage forms can be in solution, emulsion, suspension, capsules, tablets, ointment, gel, cream, lotion and may be given to cancer patients by injection or parenteral administration.

Because the platinum compounds disclosed in the present invention are ionic, they are more soluble in water than cispatin, carboplatin, or oxaliplatin. Therefore, it is much easier to formulate these compounds into suitable pharmaceutical dosage forms as compared to cisplatin, carboplatin, and oxaliplatin (these three are associated with low water-solubility).

Therefore, the present invention discloses pharmaceutically acceptable dosage forms of the platinum compounds. Each of the dosage form comprises one or a plurality of pharmaceutically acceptable excipients. Said excipients comprise one or a plurality of the following: water, alcohol, mannitol, lactose, sorbitol, glucose, starch, sodium chloride, sodium saccharine, cellulose, calcium carbonate, silicon dioxide, talc, polyvinyl pyrrolidone, sodium starch glycolate, sodium carboxymethyl cellulosemethycellulose, stearic acid, magnesium stearate, citric acid, sodium citrate, methyl paraben and propyl paraben.

The method for treating cancer further comprises treating the cancer patient with a further cancer therapeutic agent. Said further cancer treating agent comprises radiation, which is selected from the group consisting of X-ray radiation, UV-radiation, γ-radiation, or microwave radiation.

In addition, the platinum compounds may also be used in the treatment of AIDS (Acquired Immune Deficiency Syndrome). Because of the potential ability of these compounds to hamper the DNA or RNA replication process, it is likely that these compounds are effective against the HIV (Human Immunodeficiency Virus) and may be used for the treatment of AIDS. Because the platinum(II) ion may be camouflaged by the ligands described in the present invention, theses platinum compounds are less likely to cause the self defense of the HIV. Thus, these platinum compounds may be used to treat AIDS.

Therefore, it is also disclosed a method of treating Acquired Immune Deficiency Syndrome (AIDS) comprising administering orally or parenterally to an AIDS patient a therapeutically effective amount of the compound in claim 1 in a pharmaceutically acceptable dosage form.

### SUMMARY, RAMIFICATION, AND SCOPE

In conclusion, a series of novel platinum compounds and methods of treating cancer are disclosed in this invention.

Although the description above contains many specificities, these should not be construed as limiting the scope of the invention but as merely providing the illustrations of some of the presently preferred embodiments of this invention. Thus the scope of this invention should be determined by the appended claims and their legal equivalents, rather than by the examples given.

1. A platinum compound formed as an adduct between cisplatin and one of the following ligand at a 1:1 mole ratio:

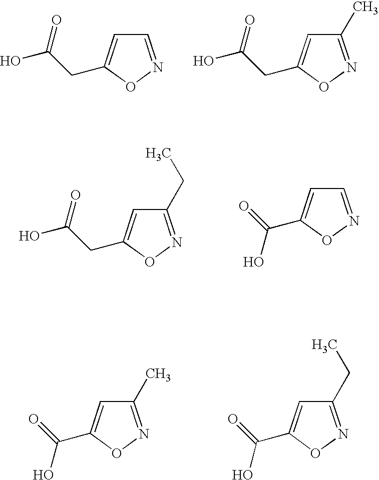

2. A method of treating cancer comprising administering to a cancer patient a therapeutically effective amount of the compound in claim 1 in a pharmaceutically acceptable dosage form.

3. The method according to claim 2 wherein said cancer comprises colon cancer, ovarian cancer, cervical cancer, breast cancer, lung cancer, head and neck cancer, brain cancer, liver cancer, leukemia, pancreatic cancer, skin cancer, prostate cancer, and stomach cancer.

4. The method according to claim 2 wherein said pharmaceutically acceptable dosage form comprises the platinum compound and suitable excipients.

5. The method according to claim 2 wherein the dosage form is for oral or parenteral administration.

6. The method according to claim 2 further comprises treating the cancer patient in combination with additional anticancer drug or drugs.

7. The method according to claim 2 further comprises treating the cancer patient with other drug that reduces the side effects from chemotherapy.

8. The method according to claim 2 further comprises treating the cancer patient with a further cancer therapeutic treatment which includes X-ray radiation, UV-radiation, γ-radiation, or microwave radiation.

9. A method of treating Acquired Immune Deficiency Syndrome (AIDS) comprising administering orally or parenterally to an AIDS patient a therapeutically effective amount of the compound in claim 1 in a pharmaceutically acceptable dosage form.

10. A platinum compound having the following formula 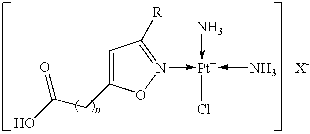
wherein n=0 or 1, R is H, CH₃, or CH₂CH₃, and X is Cl, Br, or NO₃.

11. A method of treating cancer comprising administering to a cancer patient a therapeutically effective amount of the compound in claim 10 in a pharmaceutically acceptable dosage form.

12. The method according to claim 11 wherein said cancer comprises colon cancer, ovarian cancer, cervical cancer, breast cancer, lung cancer, head and neck cancer, brain cancer, liver cancer, leukemia, pancreatic cancer, skin cancer, prostate cancer, and stomach cancer.
